(19) United States
(12) Patent Application Publication
(10) Pub. No.: US 2005/0096577 A1
(43) Pub. Date: May 5, 2005

(54) Pedi protector

(76) Inventors: Natasha LaQuelle Sykes, Lawrenceville, GA (US); Rosa Lee Worthy, Fayetteville, GA (US)

Correspondence Address: Natasha L. Sykes, 1028 Post Horn Run, Lawrenceville, GA, 30045, US

(21) Appl. No.: 10/699,030

(22) Filed: Nov. 3, 2003

(51) Int. Cl. A61F 5/00

(52) U.S. Cl. 602/30

(57) ABSTRACT

Pedi Protectors are clear lightweight plastic (cellophane) covers designed to protect the nail polish on toes while wearing shoes. The plastic is lined with lightweight oil such as Vitamin E oil. The oil acts as a lubricant on the toes to protect the nail polish from chipping or smudging. The oil also conditions the skin on the toes, leaving them smooth and moisturized. The Pedi Protector cover cladding is attached to cover 1¼ inch past the base of the toes, to the skin with a strip of type hypoallergenic adhesive type tape that holds the Pedi Protectors in place comfortably without irritation. The Pedi Protectors cladding and interior is a very lightweight piece and can be worn everyday to extend the life of a pedicure.

### CROSS-REFERENCE TO RELATED APPLICATION

The use of cover protection mainly for foot and Pedicure protection are known in particular types of numerous devised and utilized inventions that consist of many known prior art which have fulfilled certain objectives and requirements.

U.S. Pat. No. 6,415,795 Invention relates to a foot cushion pertains to a new protective foot covering inventive device that includes a sleeve being adapted to fit about a foot of a user.

U.S. Pat. No. 6,234,988 Invention relates to an improved energy absorbing system for clothing and protective gear, Worn to support and protect the wearer of socks to protect the ankle.

U.S. Pat. No. 6,196,986 Invention relates generally to foot protectors for use in playing soccer on the sand. The sock of the invention may be constructed as a sock within a sock.

U.S. Pat. No. 6,298,496 Invention relates to protective sock used for the foot and entire toe area including the ankle.

U.S. Pat. No. 5,644,813 Invention relates generally to foot floor cleaning devices, it relates to a disposable overshoe mop.

U.S. Pat. No. 5,186,189 Invention relates to fingernail polish protector a finger-encircling piece, elongated pieces extending in parallel from opposing sides of the finger-encircling piece.

U.S. Pat. No. 4,139,003 Invention relates to the field of protective coverings for injured areas of an individual and also relates to a covering designed to provide a protection to cast or bandage area.

U.S. Pat. No. 4,562,834 Invention relates to waterproof coverings for human limbs.

U.S. Pat. No. 4,639,945 Invention is a unique and improved shield to protect a limb, which has been injured.

U.S. Pat. No. 5,230,333 A thermal sock includes on the top part of the toe portion of the sock a pocket for receiving a chemical heating pouch

U.S. Pat. No. 5,623,734 Invention relates to a pedicure sock for keeping a foot warm during a pedicure.

5,906,007 Invention relates to an article of footwear for use during and following the application of a pedicure.

With perspective to the protective coverings and covering according to the present inventions it substantially departs from the conventional concepts and designs of the prior art, and in so doing provides an device primarily developed for the purpose of preventing pedicure polish nails from chipping or smudging.

The inventive device includes a lightweight plastic cover intended to cover 1¼ inch past the base of the toes then cuffs the forefoot starting from the base of the toes moving up and around the frame of the toes. The inside lining contains a liquid substance, which has a type lubricant surrounding inside the lining of the Pedi Protector.. A strip type adhesive is attached around the opening of the Pedi Protector to connect, secure, or wrap to the skin.

Requirement for obtaining a patent is if the idea is nonobvious and if it would be viewed as an unexpected or surprising development also all previous inventions in the field of an invention for which a patent is being sought, with thorough research of prior art the idea has to be sufficiently unique and non intuitive and will qualify for patent protection. This invention is physically different in some way from all previous Prior Art.

### STATEMENT REGARDING FEDERALLY SPONSORED RESEARCH OR DEVELOPMENT

Not Applicable

### REFERENCE TO SEQUENCE LISTING, A TABLE OR A COMPUTER PROGRAM LISTING COMPACT DISK APPENDIX

Not Applicable

### BACKGROUND OF THE INVENTION

This field of invention relates to a new and useful Pedicure Protection, specifically to such Pedicure Protectors, which are used for pedicure of toenail to prevent chipping or smudging polish that is applied.

Many salons, spas, and in-home pedicures are being done everyday; consumer's pay greatly for pedicures in many cases once the pedicure is complete the protection for preserving this service is not available. The objects and advantages of the pedicure protection described below for this invention are:
To provide a pedicure protection which can be produced in large quantities at a low price.
To provide a pedicure protection which both is flexible and light, and easy to use.
To provide a protection for pedicure toe nails to last for longer period of days.
To provide a new product that protection is focus only to keep pedicure nails from chipping or smudging.

Further objects and advantages are to provide a functionally design that is built with preventive inner lining, that create a environment of lubricant on the toes while wearing shoes. The lubricate also conditions the skin on the toes, leaving them smooth and moisturized.

### BRIEF SUMMARY OF THE INVENTION

The undersigned has invented a new and useful process, article of manufacture, design entitled Pedi Protector, an clad type cover protection for pedicure nails prevents polish applied from chipping or smudging. The Pedi Protector is a lightweight plastic cover designed to protect the nail polish on the toenails while wearing shoes. The Pedi Protector is intended to cover 1¼ inch past the base of the toes. The Pedi Protector clad cuffs the foot starting from the base of the toes moving up and around the frame of the toes. The inside has a lining that contains a liquid substance, which produces a lubricant surrounding. A type adhesive strip is attached around the opening of the Pedi Protector to connect, secure, and wrap to the skin.

### BRIEF DESCRIPTION OF THE SEVERAL VIEWS OF THE DRAWING

FIG. 1 is a exploded detailed view of the new pedi protector fore foot covering according to the present invention.

FIG. 2 is a Full View of the present invention

FIG. 3 is a Side View of the present invention

FIG. 4 is a Top View of the present invention

FIG. 5 is a Front Page View of the present invention

### REFERENCE NUMERALS IN DRAWINGS

10 reference the pedi protector

20 reference inside lining of the pedi protector

30 reference the closure adjustable strap

40 reference cover of the pedi protector

### DETAILED DESCRIPTION OF THE INVENTION

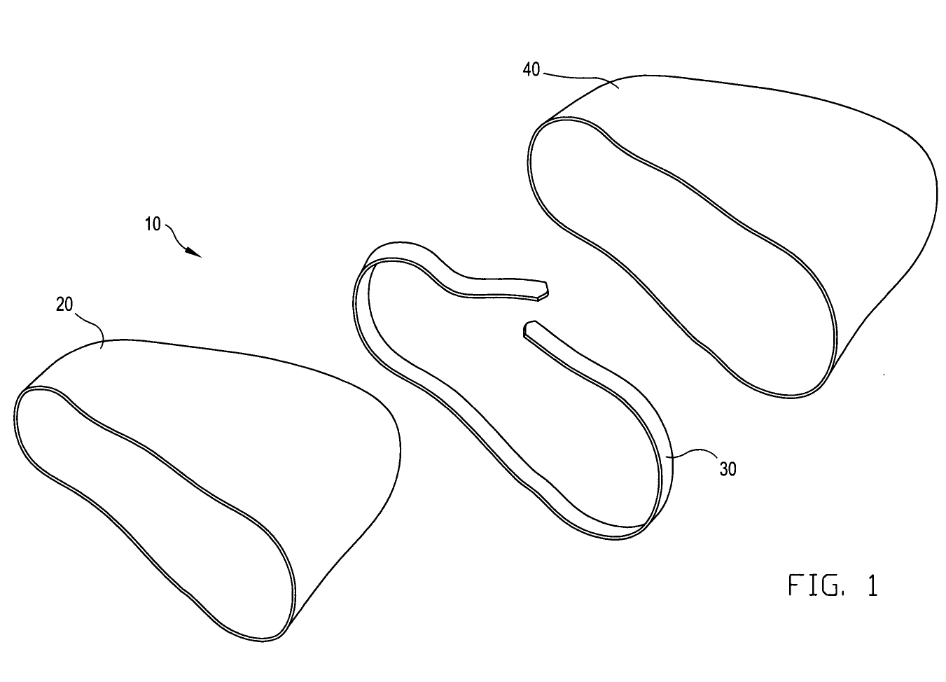

FIG. 1 exploded detailed view of the new pedi protector fore foot clad covering according to the present invention. Numeral 10 reference the pedi protector as exploded detail view numeral 40 references the cover clad piece of the pedi protector, numeral 20 reference inside lining clad piece of the pedi protector numeral 30 reference the closure adjustable adhesive type strap piece.

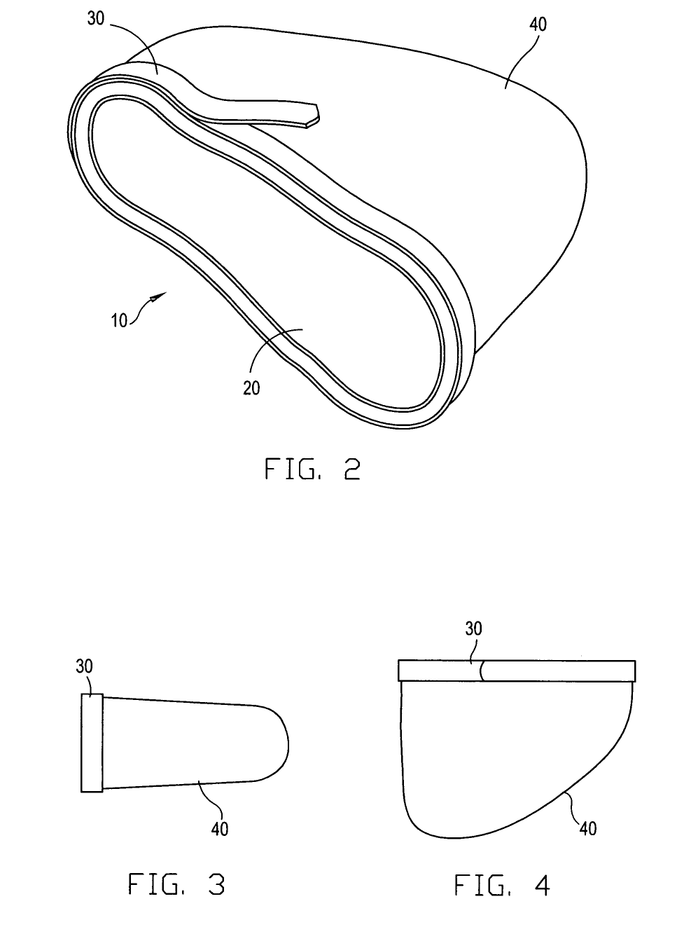

FIG. 2 is a Full View of the present invention numeral 10 reference the pedi protector showing ref 40 the cladding cover piece fitted, with ref 20 inside piece lining of the cladding of the pedi protector, sealed together permanent to hold a type liquid creating clad enclosed cover. Numeral 30 references the closure piece permanent secured leaving adjustable adhesive type strap connected to ref 40.

FIG. 3 is a Side View of the present invention showing side view of the ref 30 strap and the side view of ref 40.

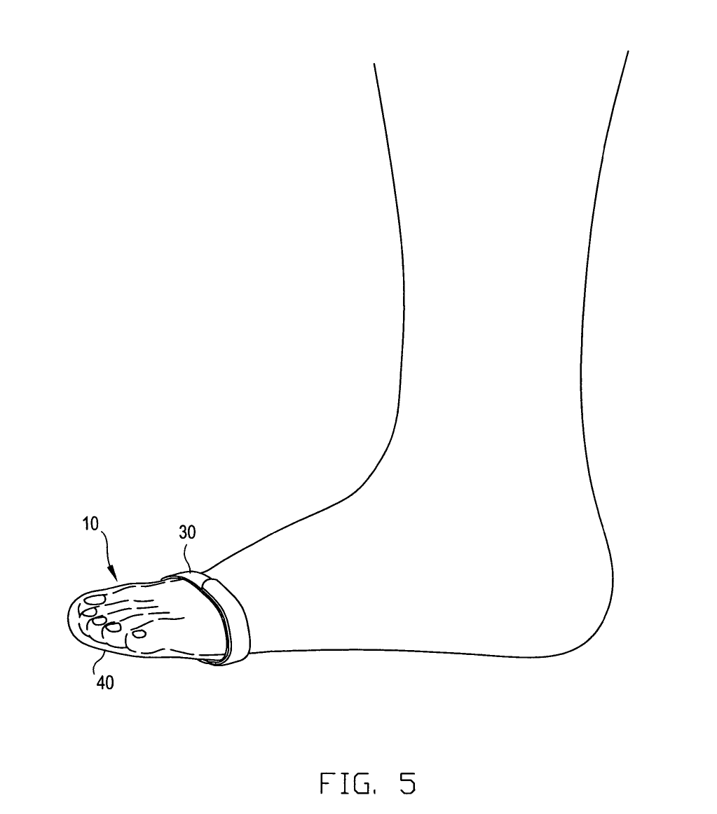

FIG. 4 is a Top View of the present invention showing top view of the ref 30 strap closure and the top view of ref 40 showing the clad design of the pedi protector.

FIG. 5 is a Front Page View of the present invention in use showing ref 30 closure function to fit the size of the foot ref 40 the cover clad fitted piece enclosed with type substance inside the enclose fitted piece.

1. In a protective pedicure covering for the pedicure toes of the foot: the said protective covering comprise as a cladding for encompassing toes of a foot user, and lining surrounding inside the cladding device for protective covering of said toes of a foot user. Wherein said lining surrounding inside of the cladding closure, is a material (cellophane) plastic such as vinylidene chloride polymer, filled with liquid (light weight oil), said lining surrounding the inside of said cladding sealed by adjoining the lining surrounding within the cladding cover, holding in the said liquid within the said lining surrounding the inside of the cladding cover. Said cladding cover material that hold liquid without leakage, with said cladding cover rim to encompass tab enclosure for attaching and encircling of the toes of a foot to fit.

2. The protective pedicure cover of claim 1, wherein said cladding protective cover pedicure toes encircling a tight fit around up to cover 1¼ inch past the base of the toes.

3. The protective pedicure cover of claim 1, wherein said lining surrounding inside the cladding devise creating an sealed interior layer.

4. The protective pedicure cover of claim 1, said material used for said lining and said cladding, said material such as vinylidene chloride polymer or of type, to hold said liquid inside the said sealed interior surrounding inside the cladding with no leakage.

5. The protective pedicure cover of claim 1 said cladding protective cover closure edge strip encompass type adhesive tab enclosure for securing, attaching and encircling of the toes of a pedicure foot to fit.
